(19) United States
(12) Patent Application Publication
(10) Pub. No.: US 2023/0178233 A1
(43) Pub. Date: Jun. 8, 2023

(54) BIOMECHANICS ASSESSMENT SYSTEM AND BIOMECHANICAL SENSING DEVICE AND BIOMECHANICAL ASSESSMENT PLATFORM THEREOF

(71) Applicant: SIPPLINK Technology Corporation, Hsinchu City (TW)

(72) Inventor: Kuei Ann WEN, Hsinchu County (TW)

(21) Appl. No.: 17/882,369

(22) Filed: Aug. 5, 2022

(30) Foreign Application Priority Data
Dec. 7, 2021 (TW) 110145761

(51) Int. Cl. G16H 40/67; A61B 5/00; A61B 5/11; G16H 50/30

(52) U.S. Cl. CPC G16H 40/67; A61B 5/0022; A61B 5/742; A61B 5/1123; G16H 50/30; A61B 2562/0219; A61B 2560/0475; A61B 2562/0223

(57) ABSTRACT

A biomechanics assessment system comprises at least one biomechanical sensing device communicatively connected to a biomechanical data interpretation device via at least one intermediate device and. The biomechanical data interpretation device is embedded in a server computer connected to the Internet and is provided with at least one biomechanical data interpretation program, each being configured to perform marking a feature, marking reference information, including one of physical activity, type of action, sensor position, sensing time and stage of sensing/action; and to perform normalization on the biomechanical data. at least one, on the biomechanical data.

### TECHNICAL FIELD

The present invention relates to a biomechanics assessment system and a biomechanical sensing device used in the system and a biomechanical assessment platform using the biomechanical sensing device, in particular to a biomechanics assessment system that uses general-purposed motion sensing devices in obtaining biomechanical data and uses artificial intelligence assisted biomechanical assessment platform in collecting various physical activity-related biomechanical data and in the assessment of biomechanical data.

### BACKGROUND OF THE INVENTION

In Taiwan, the main causes of traumatic spinal cord injury are: severe injuries and crushing, falling from height, car accidents, sports injuries, and gunshot wounds. The main causes of non-traumatic spinal cord injury are: tumors, inflammation and vascular malformations. According to European and American epidemiological statistics, the incidence of spinal cord injury is about one-thousandth of the total population. Based on this estimate, there are at least 23,000 people with spinal cord injuries in Taiwan, and about 1,000 to 1,200 are added every year. The incidence of spinal cord injury is highest between the ages of 20 and 29, accounting for about two-thirds. The ratio of men to women is about four to one.

About 3 million people in Taiwan are suffering from joint disease. Among them, there are close to 100,000 patients with knee disorders, and more than 10,000 people undergo artificial knee replacement surgery each year.

Epilepsy is a fairly common disease. According to research statistics, about 5-10 out of every 1,000 people suffer from epilepsy, so Taiwan's existing epileptic population is about 100,000 to 200,000.

In terms of dementia, according to the demographic data released by the Ministry of the Interior, Taiwan, at the end of 2019, there are 3,607,127 elderly people over 65 in Taiwan, of which 654,971 people with mild cognitive impairment (MCI), accounting for 18.16%; and 280,783 people with dementia, accounting for 7.78%.

In addition, statistics at the end of 2020 show that Taiwan's running population is approximately 1 million. The rehabilitation population is approximately 770,000.

These populations all have a common need, which is biomechanical assessment. The so-called biomechanical assessment mainly refers to the assessment of physical activities, which involves analyzing the user's physical activity description data as a reference for diagnosis, training, and rehabilitation. It usually includes evaluating the cause, degree, and improvement methods of dyskinesia, as well as the improvement process.

In order to assist physicians, technicians, coaches and other professionals to achieve the assessment of physical activities, there are a variety of physical activity assessment systems or devices that use multi-sensor motion recognition technology, which are already on the market. These devices or systems usually use a plurality of various motion sensing devices, such as inertial measurement unit (IMU) for detection to obtain the description data of a user's physical activities, supplemented by certain motion models generated by artificial intelligence deep learning to identify the user's physical activities and to analyze and evaluate the user's physical ability. In addition, according to the action guidelines designed for the diagnosis, rehabilitation, and fitness process, the evaluator can perform corresponding physical activities to achieve real-time and interactive monitoring and assessment.

Patent publication US2004/0181129A1 disclosed a subjectalized fitness diagnostic and assessment system. The system comprises a handheld electronic device, which can receive physical health indicators and calculate according to formulas to obtain an output indicating physical health.

Patent U.S. Pat. No. 7,980,997B2 relates to a system for encouraging users to perform substantial physical activity. The system includes sensors wearable during substantial physical activities such as running or playing basketball. The sensors can detect the intensity of physical activity and provide data related to the physical activity to a processing system. The processing system may display rewards to encourage the user to perform physical activities, and the rewards provided may be based on the user's physical activity.

Patent publication US2016/263439 relates to an automatic assessment device for physical activity data based on exercise. The device receives a variety of measurement result data, compare the data with stored reference values, and generate a training plan. The measurement data that the device can process include: turnover parameter such as stride rate, cadence, and stroke rate, biomedical parameter such as ECG and BP, and biomechanical parameter such as vertical oscillation, leg power balance, arm power balance, power through range of motion, footstrike impact, time on ground, and footstrike pattern.

Patent publication US2013/131846 relates to a disease treatment game device, which can produce game images and sounds, and play games with disease treatment functions. The patient's actions are detected by motion sensors and provided to the game device; whereby corresponding screens are displayed.

Patent document CN109561837A discloses a system and method for assisting physical exercise. The system detects at least two of the following parameters: i) speed, heart rate and heart rate variability, ii) running dynamics, iii) footstrike, iv) posture, and v) electromyogram (EMG) related parameters, to determine the fatigue of the wearer of the sensor.

U.S. Pat. No. 10,314,520B2 discloses a system and method for characterizing biomechanical activities, which are used to collect a set of kinematic data streams from an activity tracking system. The sensor device used is an activity tracking device with at least one inertial measurement unit.

Patent publication US2017/0095692A1 discloses a system and method for tracking running activities, including an activity tracking device and a communication module. The processor generates biomechanical signals according to the sensing data of an inertial measurement unit in the tracking device. The application can be operated on a second computer device different from the activity tracking device. The system provides a variety of communication modes and biomechanical signal generation modes.

JP2016/532468A discloses a system that uses a conformal sensor for detection and analysis of data indicating physical activities sensed by the sensor, for use in training or clinical purposes. The conformal sensor senses or measures movement (including body movement and/or muscle activity), heart rate, electrical activity, and/or body temperature.

US2018/360368A1 discloses a system and method for assessing and treating neurological deficits by analyzing voluntary and involuntary neuromuscular activity of a patient. The patient is required to perform certain prescribed physical and cognitive skills program to obtain data needed for remote assessment and treatment of the patient. The system uses a large number of detection devices, including different types of motion sensors and pressure sensors, to obtain the required data for the assessment.

U.S. Pat. No. 10,750,977B2 discloses a medical assessment system. The system uses sensors embedded in the mobile phone to sense the user's movement and collect the user's biological data. The system creates an application program to determine the user's health status based on sensor data. The biomarkers generated can represent the state or the progression of a medical condition of the patient.

From the prior art it can be found that there is a strong demand for the tracking, assessment and counseling of body activities in the market. Many businesses have developed a variety of devices and systems to meet the needs of consumers. However, existing products usually need to use a variety of sensors and detectors in one system. Sensors designed and manufactured for specific purposes cannot be used in the collection of data for different purposes.

Therefore, there is a need in the industry for a novel biomechanical assessment device and system, especially a physical activity assessment device and system, which can achieve a variety of biomechanical assessments using a generally purposed sensing device.

There is also a need in the industry for a physical activity assessment device and system that can work for a longer period of time and continue collecting biomechanical data for assessment purposes.

Meanwhile, there is also a need for a biomechanical assessment platform that can collect a wide variety of biomechanical data for long-term tracking, training, diagnosis, and analysis.

### OBJECTIVES OF THE INVENTION

A purpose of the present invention is to provide a biomechanical sensing device with a simple structure, which is easy to manufacture and can sense a variety of biomechanical activities and generate useful sensing data.

Another purpose of the present invention is also to provide a biomechanical information platform, which collects various and large amounts of biomechanical data from biomechanical sensing devices that has minimum sensing functions.

The biomechanics assessment system according to the present invention comprises at least one biomechanical sensing device, at least one intermediate device and a biomechanical data interpretation device. The at least one biomechanical sensing device is communicatively connected to the biomechanical data interpretation device via the at least one intermediate device. In some embodiments, the biomechanical data interpretation device can be embedded in the intermediate device, in particular, in the form of application software. The biomechanical data interpretation device can also be embedded in a server computer connected to the Internet in the form of application software. The biomechanical data interpretation device may be built in the server computer to form a biomechanical assessment platform, which serves to communicate with a great number of biomechanical sensing devices for uploading biomechanical data thereto and to communicate with computer devices in connection with the platform, for utilization of the biomechanical data and biomechanical information stored therein, such as processing the biomechanical data using the biomechanical data interpretation device/software and downloading various processing result information.

The biomechanical sensing device comprises at least one three-axis inertial sensor for sensing the movement of the biomechanical sensing device and outputting the sensing data; an interface device for receiving user input for setting at least one format for output data of the biomechanical sensing device; a wireless communication device for establishing a communication channel with the at least one intermediate device for exchange of data; and a power supply for supplying electric power to the sensor, the interface device and the wireless communication device. In a preferred embodiment of the present invention, the biomechanical sensing device is configured to continuously output the sensing data via the wireless communication device in the at least one format for a predetermined time.

In a preferred embodiment of the present invention, the biomechanical sensing device may further comprise a gyroscope and/or a three-axis magnetometer.

In the preferred embodiments of the present invention, the biomechanics assessment system comprises a plurality of biomechanical sensing devices, a plurality of intermediate devices, and at least one biomechanical data interpretation device. At least one of the plural biomechanical sensing devices is communicatively connected to the biomechanical data interpretation device via at least one of the plural intermediate devices. In this embodiment, a plurality of biomechanical data interpretation device can be installed one server computer and is connected to the plurality of intermediate device via the Internet.

In a specific embodiment of the present invention, the biomechanical sensing device may further comprise a memory device for storage of the sensing data of the inertial sensor, the gyroscope and/or the magnetometer. In this embodiment, the biomechanical sensing device is configured to continuously store the sensing data in the memory device in the at least one format for a predetermined time.

The intermediate device may be a computer device equipped with a wireless communication function, preferably a smart phone, wherein a necessary application program is installed, to establish a communication channel with at least one of the plural biomechanical sensing devices for exchange of data. The application can also be used to establish a communication channel with the biomechanical data interpretation device to exchange data. The intermediate device is configured to supply or transmit the sensing data sent by the at least one biomechanical sensing device to the biomechanical data interpretation device.

In a preferred embodiment of the present invention, the interface device of the biomechanical sensing device is built in the intermediate device. In this embodiment, the intermediate device is preferably configured to provide a setting interface, preferably a graphical setting interface, for the user to input setting parameters, and to send them to the biomechanical sensing device to change the settings of the biomechanical data sensing device, such as the format of its output sensing data. In other embodiments of the present invention, the interface device of the biomechanical sensing device is built in the biomechanical sensing device.

The biomechanical data interpretation device is provided with a memory device for storing the biomechanical data generated by the at least one biomechanical sensing device. The biomechanical data interpretation device is provided with at least one biomechanical data interpretation program, each being configured to perform at least one of the following functions on the biomechanical data: marking a feature: marking reference, such as type of physical activity, type of action, sensor position, sensing time and stage of sensing/action; and normalization.

After the biomechanical data interpretation program is executed, it can mark a feature on a biomechanical data file. The feature to be marked can be at least one of the following features: the beginning and the end of a physical activity; the transition of a stage of the physical activity; the beginning, the end, and the transition of the type of a physical action; the generation of a movement trajectory, etc.

After the biomechanical data interpretation program is executed, it can identify a corresponding type of physical activity for a biomechanical data file. The type of athletic activity to be identified may be at least three of the followings: walking, running, jumping, dancing, biking, horse riding, skiing, skating, and skateboarding.

After the biomechanical data interpretation program is executed, it can identify a corresponding action type for a biomechanical data file. The types of actions to be identified can be at least one of the followings: standing still, raising a hand, raising a leg, raising a palm, swinging an arm, swinging a leg, straight punch, slashing a hand, rising block, back elbow bumps, front elbow hit, side elbow hit, round kick, back kick, forward, back, turn, bend, side bend, back bend, forwards roll and backwards roll.

After the biomechanical data interpretation program is executed, it can determine the sensor position for a biomechanical data file. The sensor position refers to the position where the sensing device is worn on a human body when sensing and can be at least four of the following positions: upper left arm, upper right arm, lower left arm, lower right arm, left palm, right palm, left thigh, right thigh, left calf, right calf, left foot, right foot, head, neck, chest, back, waist and buttocks.

After the biomechanical data interpretation program is executed, it can determine the sensing time for a biomechanical data file. After the biomechanical data interpretation program is executed, it can normalize the sensing datas of a biomechanical data file.

The biomechanics assessment system may also comprise a display device for retrieving one or more biomechanics data file from the memory device of the biomechanical data interpretation device according to a user's instruction, and displaying the requested information in a format and form specified by the user.

In a preferred embodiment of the present invention, the biomechanical data interpretation device is configured to recognize at least one synchronization feature in the one or more biomechanical data file, and determine for each file a start time and/or end time of displaying, as well as a timing of change of display content, including a data transition frequency and a frame change frequency along the time axis, according to the synchronization feature.

The above and other objectives and advantages of the present invention can be more clearly illustrated by the following detailed description with reference to the accompanying drawings.

### BRIEF DESCRIPTION OF THE DRAWINGS

FIG. 1 shows a system schematic diagram of an embodiment of the biomechanics assessment system of the present invention.

FIG. 2 shows a block diagram of a biomechanical sensing device applicable to the present invention.

FIG. 3A is a waveform diagram of sensing data showing phases of an epileptic seizure. Shown in this figure are the readings along the time axis of the tremor amplitude of the right ankle of a patient.

FIG. 3B shows a comparison of the test result of FIG. 3A relative to the judgment of a physician.

FIGS. 4A-4D are waveform diagrams of the test results of an epileptic seizure, showing the 2-norm acceleration (“ACC”) values of the measured tremor readings at the left ankle (FIG. 4A), right ankle (FIG. 4B), left wrist (FIG. 4C), and right wrist (FIG. 4D) of a patient, with the frequency as the horizontal axis.

FIGS. 5A-5D are waveform diagrams of the test results of the physical activities during walking, showing the motion measurement results of a biomechanical sensing device worn on the left ankle of a subject.

FIG. 6 shows a decision tree for determining the type of physical actions, based on sensed results of the invented biomechanical sensing devices worn on upper center of the back and the ankles of both feet, respectively.

FIGS. 7A and 7B respectively show the test results of biomechanical sensing devices worn on both ankles when the subject is walking straight.

FIG. 8 shows the Y-axis acceleration value sensed result of a biomechanical sensing device worn on the back and the waist when the subject jumps 10 times on one foot with the left foot. As shown in the figure, the Y-axis acceleration value of the biomechanical sensing device will produce a peak value every time the left foot takes off.

9A and 9B respectively show the sensed results of the X-axis acceleration of a biomechanical sensing device worn on the back and the waist when the subject jumps 10 times with one foot with the left foot.

FIGS. 10A and 10B respectively show the sensed results of the X-axis acceleration value of a biomechanical sensing device worn on the waist of the back when the subject ran 12 times on the spot.

FIG. 11 shows the waveform of the change in height from the ground detected by a biomechanical sensing device worn on the left ankle when the subject walks 7 steps forward.

FIG. 12 shows the waveform of change in the 2-norm acceleration (ACC) value detected by a biomechanical sensing device worn on the back of the right palm during the postural tremor, with the subject's hands raised horizontally for a period of time.

FIG. 13 is a flow chart of a method for detecting epileptic seizures and phases using the biomechanics assessment system of the present invention.

FIG. 14 shows a block diagram of a biomechanical data interpretation device applicable to the present invention.

table

Table I shows the calculated ACC values and descriptions thereto, according to the embodiment of FIG. 12.

Table II shows the correlation of certain expert assessment index and the sensing data of the invented biomechanical sensing device in several physical activities related to the diagnosis, treatment, and rehabilitation of Parkinson's disease.

### DETAILED DESCRIPTION OF THE INVENTION

Hereinafter, several preferred embodiments of the biomechanics assessment system of the present invention will be described with reference to the drawings. It must be noted that, for the descriptions and illustrations of the embodiments of the present invention, the purpose is only to present the main features and possible implementation modes of the present invention in a brief manner. The scope of the present invention should extend to other embodiments that can be derived or deduced by the skilled persons in the industry.

Although it is no intended to limit this invention by any theory, the inventor found that, while there are action guidelines for biomechanics assessment system designed for diagnosis, rehabilitation, fitness, training and other processes, they are all designed for professionals, such as doctors, technicians, coaches, etc. When assessing, the professionals observe the performance of the subject with the naked eye, and make diagnosis or improvement suggestions based on their observation results. The related action guidelines may include multiple sets of body and/or limb movements, all for the evaluation of the activity ability of specific parts of the body and the ability of the brain to control movement. In addition, various widely accepted standard action guidelines have been developed for use in different fields of diagnosis, rehabilitation, and fitness. All guidelines actually include substantially the same physical actions or movements or the same series of physical actions or movements.

For example, “The unified Parkinson's disease rating scale,” also known as the UPDRS rating scale, is a scale system to longitudinally measure the development of Parkinson's disease. Taking the UPD score scale as an example, the patient is required to perform several actions including the followings during the physical activity assessment process:

0. Pre-and end actions: Sit quietly in a chair with the palm placed in the middle of the thighs.

1. Rest tremor: Sit quietly in a chair with the palms placed in the middle of the thighs for 5 seconds.

2. Posture tremor of the hands: Place the palms on the thighs for 10 seconds→Stretch the arms out in front of the body with palms down for 10 seconds→Raise the palms upward and hold still for 10 seconds→Return to the end action and hold still for 5 seconds.

3. Kinetic tremor (finger-to-nose maneuver): (right hand) reach as far as possible then touch the nose 5→times stand still for 5 seconds→(left hand) reach as far as possible then touch the nose 5 times→stand still for 5 seconds.

4. Finger tapping in rapid succession.

5. Open and close hands in rapid succession (right first, then left): Pinch action for 10 seconds→Grasping action for 10 seconds→End action for 5 seconds→Change hands or next action.

6. Rapid alternating movements of hands: Pronation-supination movements of hands, vertically and horizontally, both hands simultaneously, for 10 seconds→Hold still for 5 seconds.

7. Patient taps heel on the ground in rapid succession picking up entire leg (right first, then left): Step for 5 seconds→Stamp for 5 seconds→Stand still for 5 seconds→Change foot or next action.

8. Arising from chair.

9. Posture: Arising from a chair with hands on patient's chest→Stand erect for 5 or 10 seconds.

10. Gait: Walk 5 meters forward and 5 meters back→Stand erect for 5 seconds.

11. Posture stability: Turn left→stand for 5 seconds→Pull the patient backward for 2 times, wait for 2 seconds after the subject is stable, after the first pull, and then pull again to see if the patient falls.

These actions are not only the representative actions in the UPD rating scale, they are also common testing actions in other sports-related assessment systems, although the magnitude, frequency, and focus of the actions may not be the same.

For example, in sports assessment, the actions that coaches need athletes to perform may also include:

1. Upper extremity exercises: Including one-handed or two-handed continuous or alternating forwards, forward strikes, forward splits, side strikes, back strikes, side blocks, up blocks, down blocks, etc.

2. Lower extremity exercises: One foot or both feet continuously or alternately stretch forward, kick forward, round house back, side kick, kick back, step forward, step back, half squat, etc.

3. Coordinated movement of hands and feet: At least one of the aforementioned upper extremity actions simultaneously or sequentially performs with at least one of the aforementioned lower extremity actions.

4. Body movements: Pitch up, pitch down, turn, etc.

Every time for rehabilitation, diagnosis or exercise assessment, the physician or the coach will ask the subject to perform a variety of exercises or actions in a predetermined sequence, and observe the results with the naked eye during the process, to evaluate the observation results based on the assessment criteria obtained from experience, so to provide diagnosis or advice. The problem of this assessment method is, the criteria used in the assessment are not objective, because they are mainly based on the experience of the physician or the coach. The same action may get different assessment results and suggestions. In addition, if the physician or the coach observes mainly from a specific angle, the result may be biased. Even if it is recorded from multiple angles and played at the same time, it is not easy to observe the movement correctly and objectively.

The inventor found that a motion sensor made with the microelectromechanical technology is small in size and light in weight, and is suitable for wearing on the body to sense the motion of the body, if wireless communication capabilities are added thereon. Although the sensed results of the motion sensor are only readings and cannot be used to evaluate exercises or actions, or for other biomechanical assessments, a suited interface can be provided to convert the readings of the motion sensor into data of a format that can be interpreted by an interpretation device, into useful biomechanical information, or even into a three-dimensional representation of graphics for assessment and suggestions by physicians or coaches.

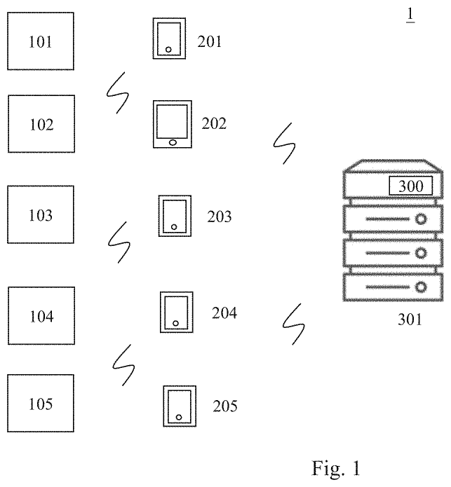

Based on these discoveries, the present invention provides a biomechanics assessment system, comprising at least one biomechanical sensing device, at least one intermediate device, and a biomechanical data interpretation device. FIG. 1 is a system schematic diagram showing an embodiment of the biomechanics assessment system of the present invention. As shown in the figure, the biomechanics assessment system 1 includes a plurality of biomechanical sensing devices 101-105, a plurality of intermediate devices 201-205, and a biomechanical data interpretation device 300. Among them, each biomechanical sensing device 101-105 are communicatively connected to the biomechanical data interpretation device 300 via any one intermediate device 201-205. In some embodiments, the biomechanical data interpretation device 300 can be embedded in any of the intermediate devices 201-205, preferably in the form of application software. However, in the preferred embodiments of the present invention, the biomechanical data interpretation device 300 is embedded in a server computer 310 connected to the Internet, in the form of application software.

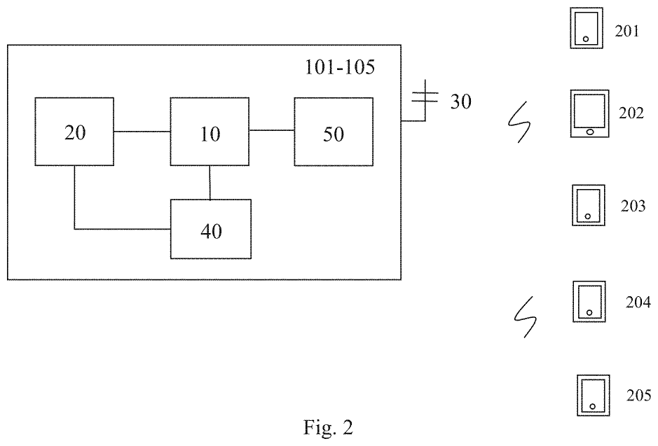

FIG. 2 shows the block diagram of one embodiment of the biomechanical sensing device 101-105 applicable to the biomechanics assessment system 1 of the present invention. The biomechanical sensing device 101 as shown in the figure includes a motion sensing element 10. The motion sensing element 10 is preferably a three-axis inertial sensor, and more preferably includes at least one of a three-axis accelerometer, a three-axis gyroscope, and a three-axis magnetometer. Preferably, it has a three-axis accelerometer and a three-axis gyroscope, or a three-axis accelerometer and a three-axis magnetometer. The accelerometer senses the motions of the biomechanical sensing device itself, and outputs the sensing data of their three-axial components. The gyroscope measures the angular velocity of the motions in the three-dimensional space and calculates the angular velocity. The magnetometer measures the geomagnetism and outputs the three-axial components of the sensing data. In most applications, only a three-axis accelerometer would be sufficient. However, as the motion sensor has become a popular commodity, motion sensing components available in the market have already provided a three-axis accelerometer, a three-axis gyroscope, and a three-axis magnetometer. Such motion sensing elements are very suitable for application in the present invention. Of course, the motion sensing element 10 applicable to the present invention is not limited to this.

The biomechanical sensing device 101 also includes an interface device 20, for accepting user input and setting a predetermined format for the output data of the biomechanical sensing device 101. The interface device 20 is preferably a graphical interface for the user to input parameters to set the type and format of the output sensing data of the biomechanical sensing device 101. The interface device 20 is connected to a storage device or a temporary storage device (to be described in detail hereinafter) for storing or temporarily storing the sensing data of the motion sensing element 10, so to use the parameter input by the user to determine a type and/or a format of the output data of the storage device or the temporarily storage device. Here, the term “type” denotes to, for example, the particular axis of an axial component of the sensing data, such as, the X-axis component of the sensing data of the accelerometer. The term “format” denotes to, for example, the sampling frequency or time resolution of the output reading.

As will be explained below, the interface device 20 does not necessarily have to be built in the biomechanical sensing device 101. In a preferred embodiment of the present invention, the interface device 20 of the biomechanical sensing device 101 is built in one of the intermediate devices 201-205 in the form of application software. In this embodiment, the intermediate device is configured to provide the setting interface, preferably a graphical human-machine interface, for ease of operation. The setting result is then provided to the biomechanical sensing device 101-105 in a wired or wireless manner.

The biomechanical sensing device 101 also has a wireless communication device 30 for establishing a communication channel with one of the plural intermediate devices 201-205 to exchange data. The wireless communication device 30 can be any small or micro wireless communication device, as long as it can communicate efficiently with the intermediate devices 201-205, in particular, transmit the sensed data to the intermediate devices 201-205. As explained below, the intermediate devices 201-205 are preferably smart phones. In this embodiment, the biomechanical sensing devices 101-105 only need to have short-distance wireless communication capabilities. In most embodiments of the present invention, the biomechanical sensing devices 101-105 communicate with the intermediate devices 201-205 via Bluetooth wireless communication channels.

According to the present invention, most biomechanical sensing devices 101-105 included in the biomechanics assessment system 1 send the sensed values to the biomechanical data interpretation device 300 through the plurality of intermediate devices 201-205, without modifications, so that all processing and interpretation of the sensed data are performed in the biomechanical data interpretation device 300. Preferably, a plurality of biomechanical sensing devices 101-105 corresponds to one intermediate device 201-205, and the intermediate device performs all communication and data exchange with the biomechanical data interpretation device 300. In this way, one intermediate device and a plurality of biomechanical sensing devices 101-105 are combined into a group, to be used by a specific group of people, such as a group of a physician and several patients, one coach and specific athletes, etc. The biomechanical data interpretation device 300 can usually be built in the cloud, and through the support of the intermediate devices 201-205, the sensing data are sent to the biomechanical data interpretation device 300 for interpretation. In this way, the biomechanical sensing devices 101-105 only need to be general-purposed motion sensors, and do not need to be equipped with specific functions. They can be used to perform various biomechanical monitoring and assessments, and provide descriptive exercise/action related information, for training, diagnosis and treatment. In such embodiments, the biomechanical data interpretation device can be embedded in one server computer and connected to the at least one intermediate device via Internet communication.

In addition, the biomechanical sensing device 101 also includes a power supply 40. The power supply 40 supplies electric power to the motion sensing element 10, the interface device 20 and the wireless communication device 30. Any power supply device can be used as the power supply 40 of the present invention. The power supply 40 may be a household power source but is preferably a battery, for making the wearer feel comfortable. The power supply 40 may include a power management chip to save power and avoid accidents.

In a preferred embodiment of the present invention, the biomechanical sensing device 101 is configured to continuously output the reading value of the motion sensing element 10 via the wireless communication device 30 in a predetermined format for a predetermined time. Although it is broadcast in form, the sensing data is provided to one specific intermediate device, only.

In other embodiments of the present invention, the biomechanical sensing device 101 may further include a memory device 50 for storing the reading value of the motion sensing element 10. In this embodiment, the biomechanical sensing device 101 is configured to continuously store the reading value of the motion sensing element 10 in the memory device 50 in the preset format within the predetermined time.

The intermediate devices 201-205 are a computer equipped with wireless communication abilities, usually a smart phone or a tablet computer. Of course, the intermediate device 201-205 can also be a computer with a special specification, equipped with necessary wireless communication function, to read or receive the sensing data from a specific group of biomechanical sensing devices 101-105, and to send the biomechanical data to the biomechanical data interpretation device 300. A smart phone is preferable, because in addition to the above-mentioned capabilities, application software for various purposes can be built in the smart phone. However, the intermediate devices suitable for the present invention are not limited to smart phones and tablet computers.

Each of the intermediate devices 201-205 is embedded with a necessary application program, for establishing a communication channel with at least one of the plural biomechanical sensing devices 101-105, for exchange of data. The main purpose of the application programs is to read, extract or receive sensing data from the biomechanical sensing devices 101-105. The application program also provides a parameter setting function, to provide the control parameters set by the user to the biomechanical sensing devices 101-105. The application program can also establish a communication channel with the biomechanical data interpretation device 300, also for exchange of data. The application program enables an intermediate device 201-205 to communicate with the biomechanical data interpretation device 300, so to supply or transmit the sensing data sent by at least one of the plural biomechanical data of the sensing devices 101-105 to the biomechanical data interpretation device 300.

As mentioned above, the interface device 20 of the biomechanical sensing device can also be built in the intermediate device 201-205. The advantage of this embodiment is that the human-machine interface of the biomechanical sensing device 101-105 can be simplified, or even omitted. Other advantages include the ability to provide a graphical parameter-setting interface on, for example, the screen of a mobile phone, which facilitates the user to input setting parameters. Since both the biomechanical sensing device and the intermediate device have wireless communication capabilities, the set parameters are easily transmitted to the biomechanical sensing devices 101-105 to set the type and the format of the output sensing data. The graphical human-machine interface can also provide the function of displaying the sensing data, so that setting of parameters and display of the sensing data can be performed on the same interface device.

In addition, also as mentioned above, in a specific embodiment of the present invention, at least one biomechanical sensing device 101-105 may also be built in one of the intermediate devices 201-205. In particular, most mobile phones are equipped with useful motion sensing components. The sensing ability of the motion sensing components may be good enough for certain biomechanical assessment tasks. Although such embodiment falls within the scope of the present invention, the stand-along biomechanical sensing devices are preferred, mainly because they are small-sized, lightweight and do not interfere with normal activities. The sensed results of the biomechanical sensing devices are provided to the biomechanical data interpretation device 300 through an intermediate device, although in some embodiments, the biomechanical data interpretation device 300 may also be built in the intermediate device.

In most embodiments of this invention, the biomechanical data interpretation device 300 is installed in a server computer, so it can be equipped with powerful computing and memory capabilities. The memory device of the biomechanical data interpretation device 300 can store biomechanical data/information generated by a large amount of biomechanical sensing device. For example, to store epileptic seizure monitoring data of 20,000 people generated in a year, 500 TB of memory capacity may be required. This capacity can be built in a small to medium enterprise server. The biomechanical data interpretation device 300 can install a variety of biomechanical data interpretation programs, each providing at least one interpretation function when in operation, and is configured to perform respective interpretation on the received/stored biomechanical data. According to the biomechanics assessment system of the present invention, the process of the biomechanical data interpretation device 300 may include: Automatically marking the received or stored biomechanical information/data, including marking features and reference information for the biomechanical data, such as marking a type of exercise, marking an action type, marking a sensor position, marking a sensing time, marking a sensed action stage etc. In addition, the biomechanical data interpretation device 300 can also automatically normalize the values of the biomechanical data.

In the following several embodiments of the application of the biomechanical sensing device in detecting several basic actions when conducting health assessment will be described, followed by introducing certain motion interpretation functions of the present invention.

### Embodiment 1: Start Time and End Time

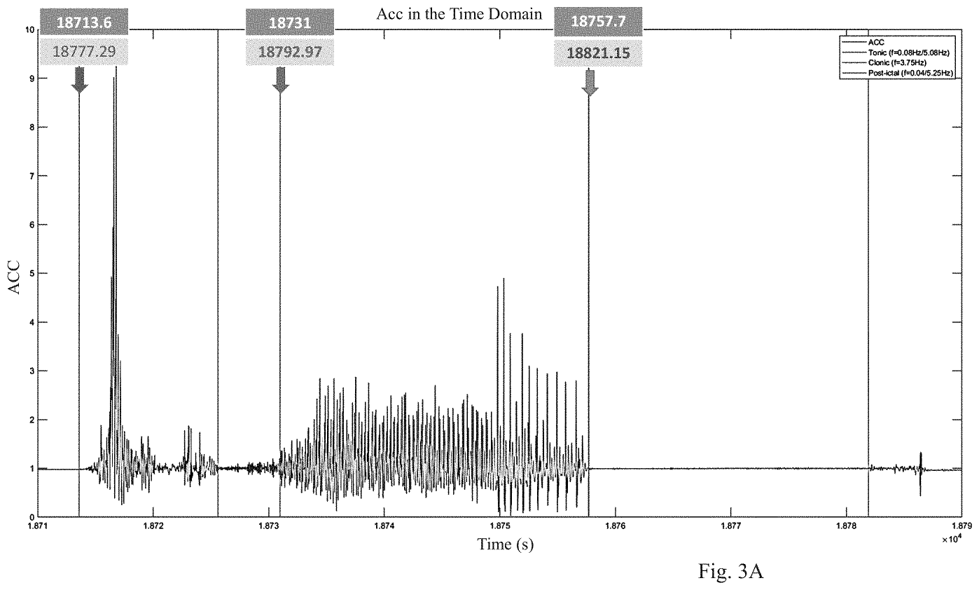

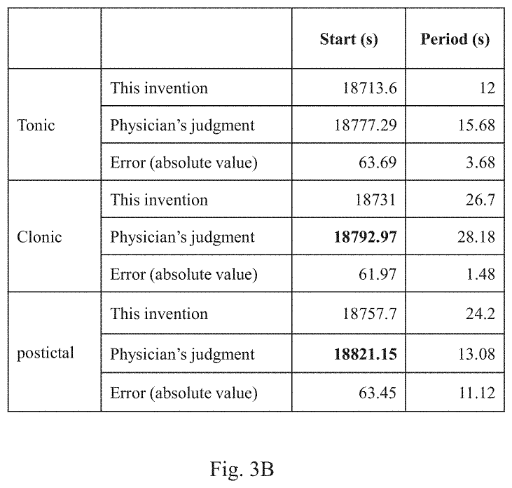

FIG. 3A is a waveform diagram of sensing data showing phases of an epileptic seizure. Shown in this figure are the readings along the time axis of the tremor amplitude of the right ankle of a patient. During the measurement, the biomechanical sensing device is placed flat on the outer side of the patient's right ankle, so that the sole of the patient's foot is in contact with the ground. Aa the waveform of the measurement result clearly shows the stages of an epileptic seizure, it is possible to write a computer program to determine the time points of the three stages of epileptic seizures, namely tonic, clonic and postictal, using the pattern recognition technology. The start time of the determination result is shown in the upper box in the figure. The number in the lower box in the figure is the start time determined by a physician. FIG. 3B shows a comparison of the test result of FIG. 3A relative to the judgment of a physician. As shown in the figure, the result of the judgment made by the pattern recognition method is close to that of the physician. It is proved that the start, transition and end times of the subject's actions can be easily identified, by simply wearing a biomechanical sensing device on the human body.

### Embodiment 2: Change of Type of Exercise or Actions

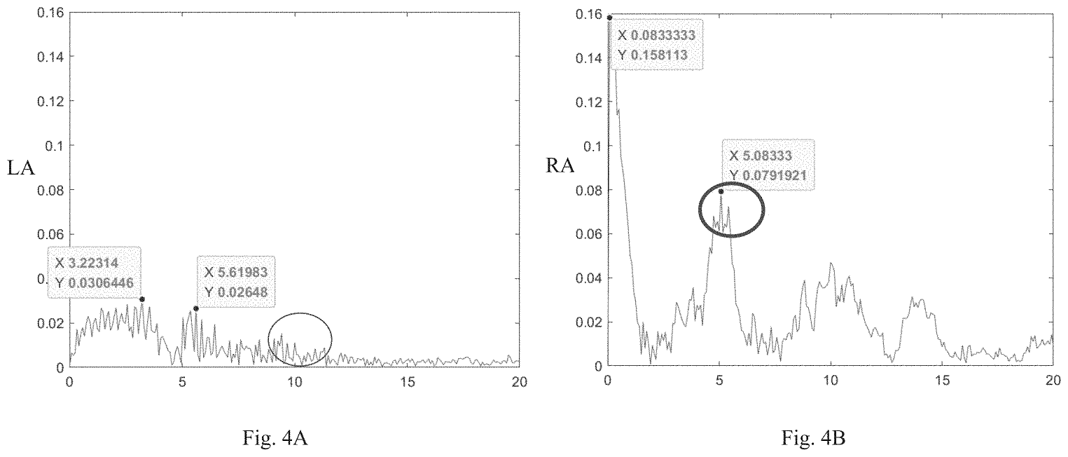

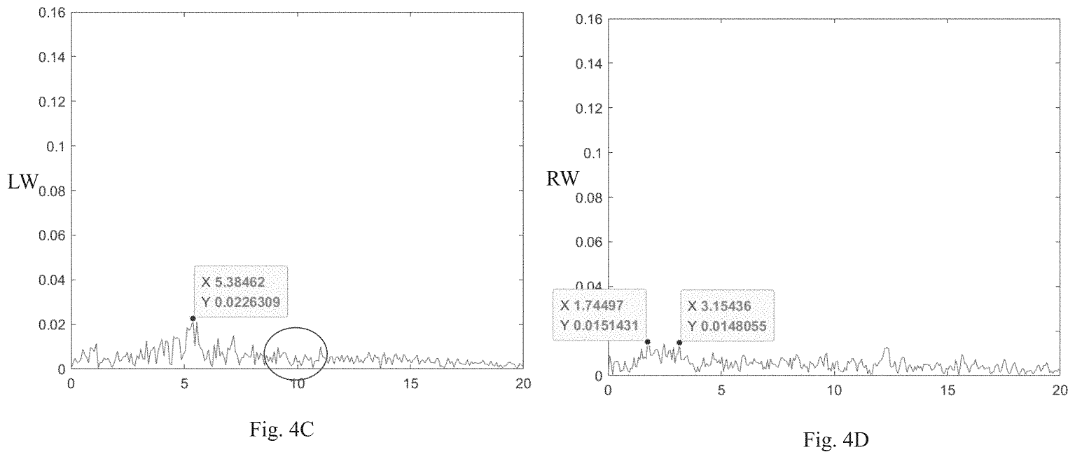

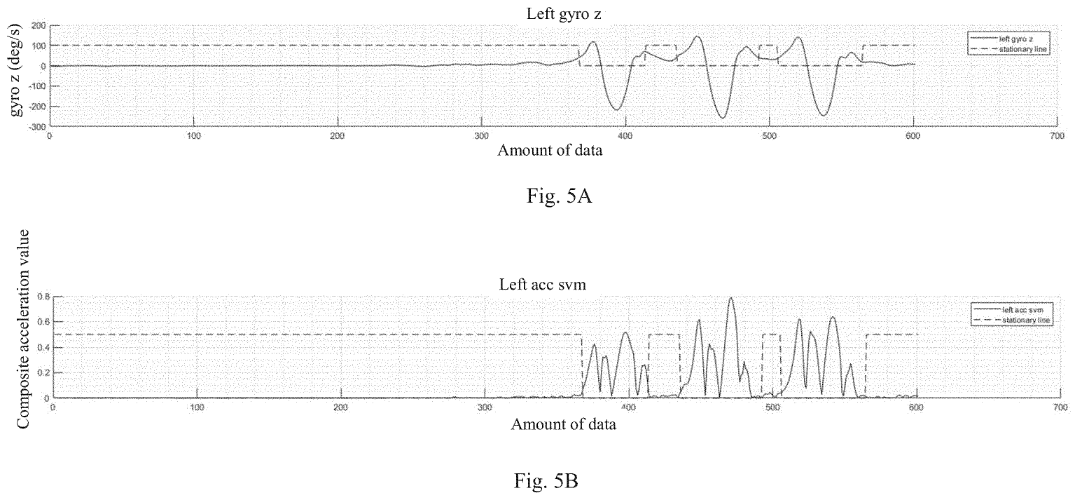

FIGS. 4A-4D are waveform diagrams of the test results of an epileptic seizure, showing the 2-norm acceleration (hereinafter “ACC,” formula to be described below) values of the measured tremor readings at the left ankle (FIG. 4A), right ankle (FIG. 4B), left wrist (FIG. 4C), and right wrist (FIG. 4D) of a patient, after spectrum analysis, with the frequency as the horizontal axis. During the test, the biomechanical sensing device is placed on the patient's ankle and wrist to record the patient's movement during admission.

ACC=\sqrt{accx²+accy²+accz²} (1)

As shown in the figure, after the frequency analysis, the results show the tonic period can be roughly regarded as a signal component of 4.5-6 Hz, especially around 5 Hz. At this time, the maximum ACC amplitude of the right ankle is RA, 0.079 but the frequency of the right wrist shows more recent noise. The ACC amplitude needs to be integrated twice to obtain the swing distance.

In addition, the measurement results in the same way found that the clonic period can be regarded as the occurrence of frequency components of 2.5-4 Hz.

The experiment shows that the change of type of actions can be detected based on the frequency analysis result of the sensed result of the biomechanical sensing device.

### Embodiment 3: Phase Detection of Action History

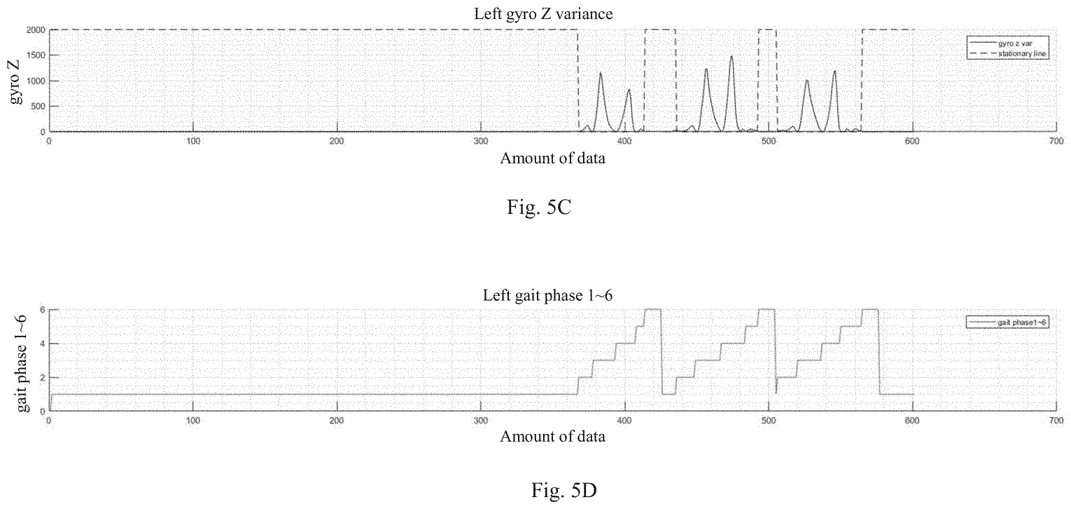

FIGS. 5A-5D are waveform diagrams of the test results of the physical activities during walking, showing the sensed result of a biomechanical sensing device worn on the left ankle of a subject, with the X-axis of the motion sensors all oriented in the direction of walking. FIG. 5A shows the Z-axis angular velocity sensed result of the gyroscope of the biomechanical sensing device. FIG. 5B is the 2-norm acceleration (ACC) value of the accelerometer of the biomechanical sensing device. FIG. 5C shows the variance of the value of FIG. 5A, which represents the variance of the Z-axis angle, useful in judging each stage of the action history (such as swing/stop). FIG. 5D shows the gait phase calculated based on the above three values, with each gait cycle being divided into 4 phases, each represented by a number.

In addition, the sensing data can be further calculated to obtain values representing the two states of standing and moving, as shown by the dashed lines in FIGS. 5A to 5C.

The experiment shows that the phase transition of an action history can be correctly identified based on the various sensed results of the invented biomechanical sensing Device.

### Example 4: Detection of Type of Actions During Exercise

If an physical exercise assessment only includes limited types of action, or limited combinations of types of action, the sensed result of the biomechanical sensing device can be used to determine the type of action performed at a specific time.

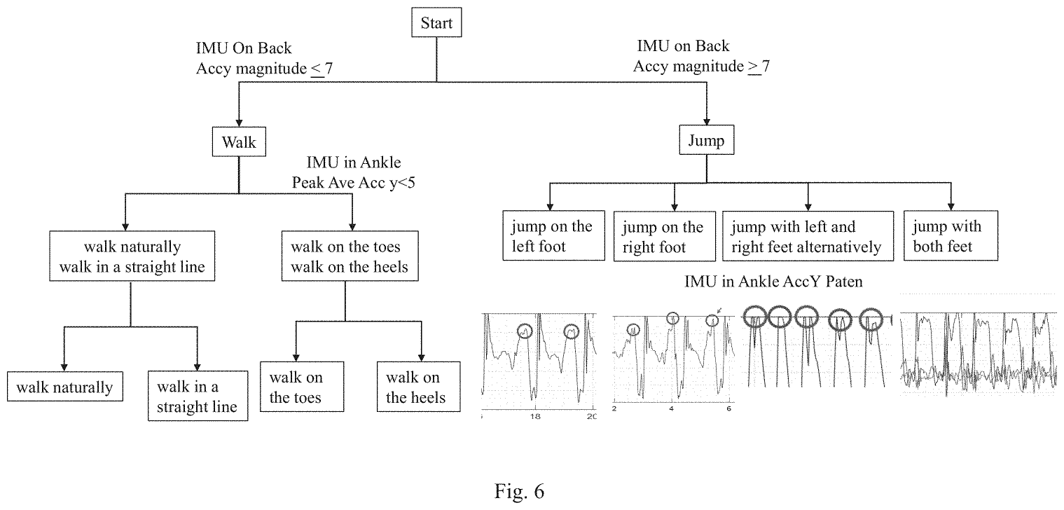

FIG. 6 shows a decision tree for determining the type of physical actions, based on sensed results of the invented biomechanical sensing devices worn on upper center of the back and the ankles of both feet, respectively, of a subject, all with the X-axis of the sensor of the biomechanical sensing device oriented in the direction of walking. As shown in the figure, in the first layer, according to the Y-axis acceleration value detected by the biomechanical sensing device on the back, the type of the action, either walking or jumping, can be determined. In this embodiment, when the Y-axis acceleration value is less than 7, it can be judged that the subject is walking; otherwise, jumping.

In the second layer, when walking, the average peak acceleration value of the Y-axis acceleration value (Acc Y), detected by the biomechanical sensing devices on the angles is used to judge how the subject is walking. In this embodiment, the value of 5 or more can be judged as the subject is walking naturally or in a straight line. On the contrary, it is judged that the subject is either walking on the toes or on the heels, as shown in the third layer on the left in FIG. 6. In addition, the peak value of the Z-axis angular velocity detected by the biomechanical sensing device worn on the ankles can be used to determine the foot that is in motion. For example, when the Z-axis angular velocity reaches −2000, it is determined that the left foot is swinging; when it reaches +2000, it is determined that the right foot is swinging.

In addition, when it is judged that the subject is jumping, the value detected by the biomechanical sensing device on his back can be used to determine whether the subject is jumping on the left foot, on the right foot, jumping with left and right feet alternatively, or jumping with both feet. For example, in this embodiment, a specific peak can be found from the waveform of the sensed result of the biomechanical sensing device on the ankle for judgment, as shown in the third layer on the right side of FIG. 6. In addition, when the slope of the X-axis acceleration value of the biomechanical sensing device worn on the back is less than 0, it is judged that the subject is jumping on the left foot; otherwise, jumping on the right foot.

This experiment shows that the type of actions during exercise can be judged based on the sensed results of the invented biomechanical sensing device.

### Example 5: Ground Time and Counts when Walking

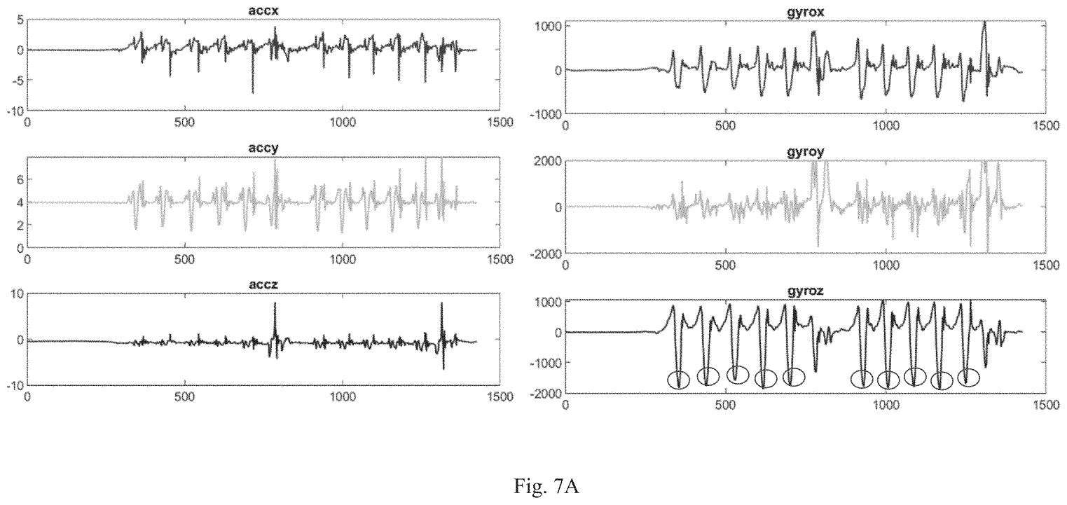

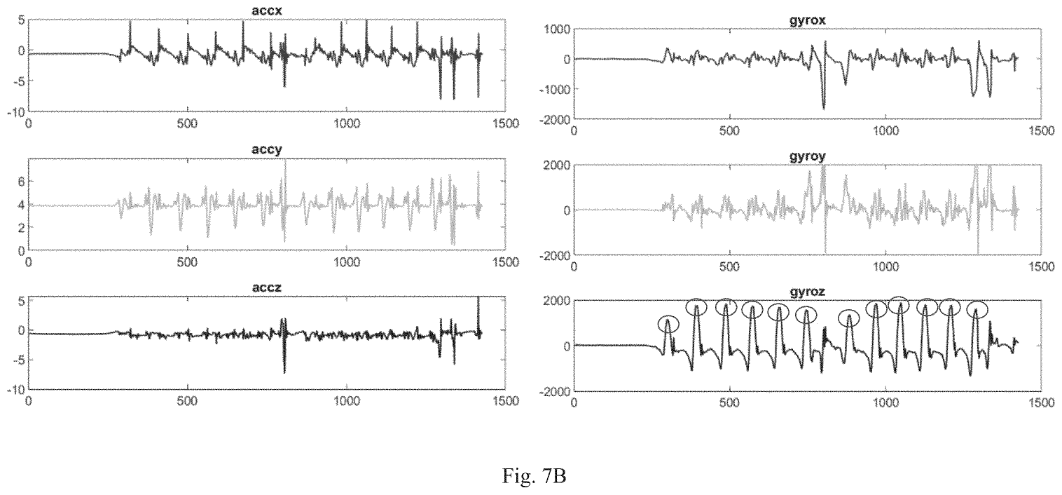

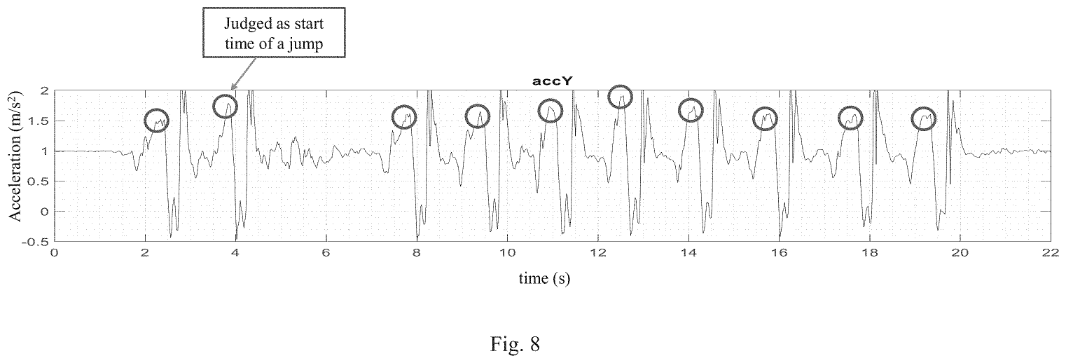

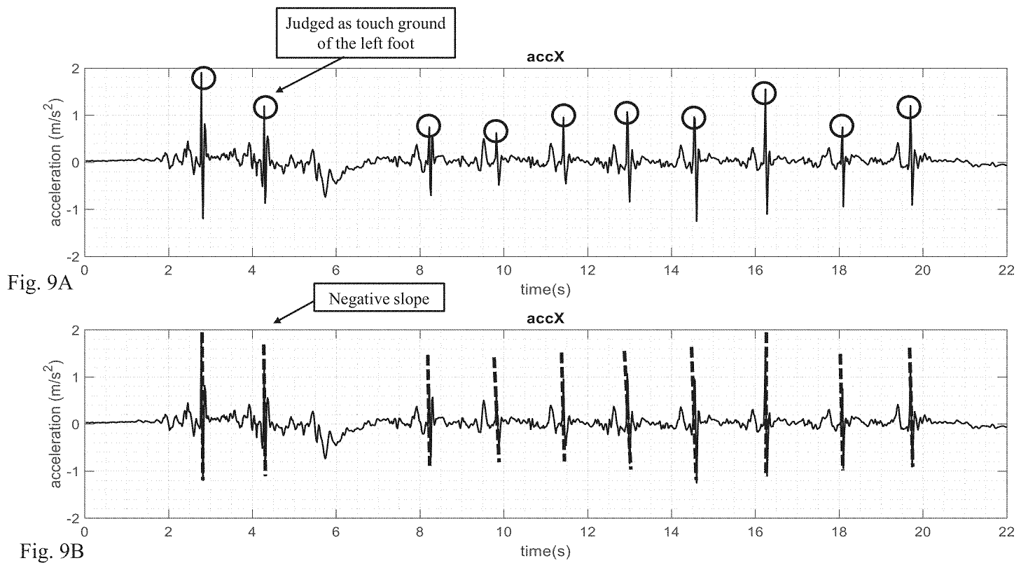

FIGS. 7A and 7B respectively show the test results of biomechanical sensing devices worn on both ankles when the subject is walking straight, wherein 7A shows the three-axis acceleration value and the three-axis angular velocity value detected by the biomechanical sensing device on the left ankle and FIG. 7B shows the three-axis acceleration value and the three-axis angular velocity value detected by the biomechanical sensing device on the right ankle. When worn, the X-axis of the sensor of the biomechanical sensing devices is all oriented in the direction of walking. As shown in the figures, the Z-axis of the gyroscope of the biomechanical sensing device worn by the left foot will produce a downward peak every time the left foot touches the ground. Conversely, every time the left foot touches the ground, the Z axis of the gyroscope by the left foot will generate an upward peak.

The waveform of the sensed result can be used to determine the time point and the counts the subject has touched the ground while walking.

### Example 6: Take-Off, Landing Time and Counts of Jumps when Jumping

FIG. 8 shows the Y-axis acceleration sensed result of a biomechanical sensing devices worn on the back when the subject jumps 10 times on one foot with the left foot. As shown in the figure, the Y-axis acceleration value of the biomechanical sensing device will produce a peak every time the left foot takes off. When worn, the Y axis of the sensor of the biomechanical sensing device orients perpendicular to the ground most of the time.

9A and 9B show the X-axis acceleration sensed result of a biomechanical sensing devices worn on the back when the subject jumps 10 times on one foot with the left foot. As shown in the figures, the biomechanical sensing device worn on the back generates a peak value of X-axis acceleration every time the left foot touches on the ground (FIG. 9A). In addition, the waveform exhibits a negative slope at the peak value, which can be used to determine that the value is the sensed result of the biomechanical sensing device worn on the back when the left foot jumps (FIG. 9B). When worn, the X-axis of the sensor of the biomechanical sensing device all faces left or right.

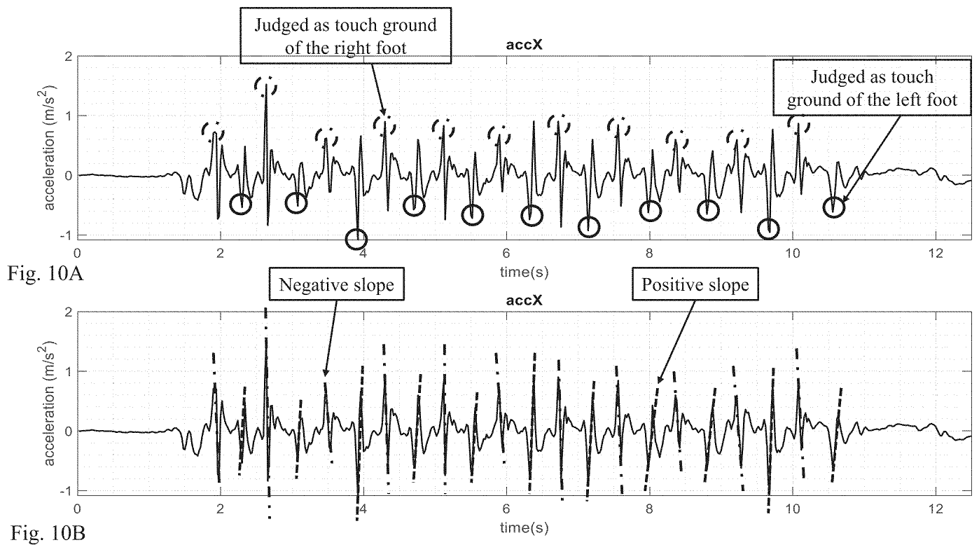

Based on the above findings, the sensed results of running 12 steps on spot were further tested. FIGS. 10A and 10B show the sensed results of the X-axis acceleration value of the biomechanical sensing device worn on the back when the subject ran 12 times on the spot. As shown in the figure, the biomechanical sensing device worn on the back generates a downward peak in the X-axis acceleration value every time the left foot touches the ground, while every time the right foot touches the ground, the X-axis acceleration value will generate an upward peak (FIG. 10A). In addition, when the waveform exhibits a negative slope at the peak, it can be judged the right foot touches the ground at that time, and when it shows a positive slope at the peak, it can be judged the left foot touches the ground at that time (FIG. 10B). When worn, the X-axis of the sensor of the biomechanical sensing device all faces left or right.

### Example 7: Motion Trajectory

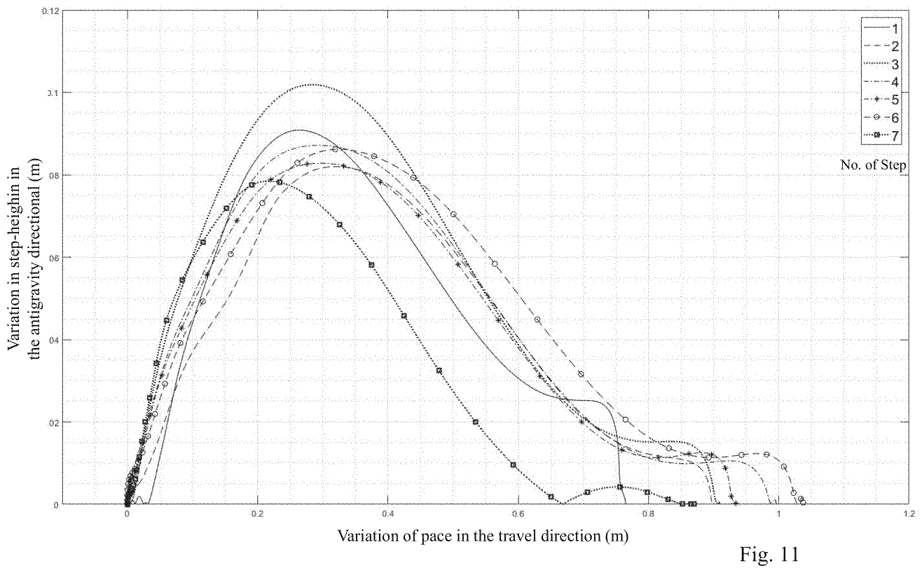

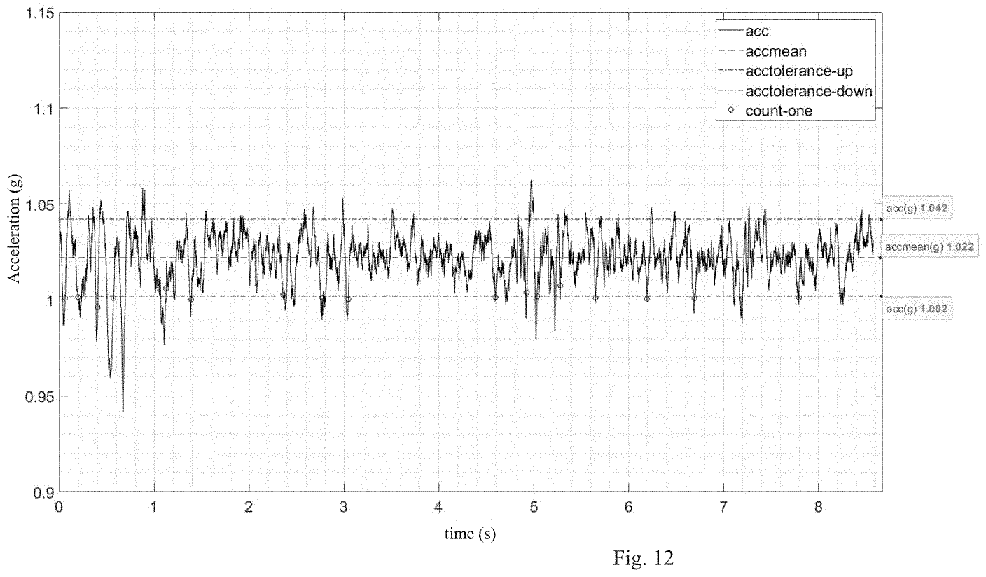

FIG. 11 shows the waveform of the change in height from the ground detected by the biomechanical sensing device worn on the left ankle when the subject walks 7 steps forward. When worn, the Y-axis of the sensor of the biomechanical sensing device faces the north direction of the earth. The integral value of the Y-axis sensed results of the biomechanical sensing device at times represents the height of the biomechanical sensing device from the ground and can be used to generate the plot of FIG. 11.

As shown in FIG. 11, the gait images of the subject's walking can be animated to be evaluated by professionals. To generate animation, even 3D animation, using the sensing data of the invented biomechanical data sensing device in plural axis, after necessary process as above, is already a mature technology. Experts in this industry can easily use commercially available hardware and software products to achieve this conversion. The technical details are thus omitted.

### Example 8: 2-Norm Acceleration ACC

FIG. 12 shows the waveform of the 2-norm acceleration (ACC) value detected by the biomechanical sensing device worn on the back of the palm of the right hand during the test of posture tremor when the subject's hands are raised horizontally for a period of time. When worn, the X-axis of the sensor of the biomechanical sensing device all the times orients the direction of the fingertip. As shown in the figure, the mean ACC value is not used as the baseline in the calculation, but a threshold is used for calibration. In this embodiment, the threshold is set to 0.02. The waveform passing through this point is recognized as a shaking when it passes through this point again. Each time it passes the threshold point twice from two directions, it is judged as one tremor count. The shaking period is the interval between two shakings, that is:

Shaking frequency=1/shaking period (2)

The calculation results of FIG. 12 can be further calculated using Formula (2), to obtain the values in Table I below. In addition, the calculation results of values detected by the biomechanical sensing device worn on the back of the palm of the right hand for a predetermined time when the subject's hands are bent and held flat is added to the table. Among them, the amplitude expresses the amplitude of the shaking motion, and the average value is used to avoid the influence of noise:

TABLE I
Calculation results using ACC values and descriptions
according to the embodiment of FIG. 12

| | | | |
|---|---|---|---|
| | Numerical value | Numerical value | |
| Analysis | (hands raised | (hands bent and | |
| parameter | horizontally) | held flat) | Definitions |

| | | | | | |
|---|---|---|---|---|---|
| Shaking | 1.7405 | Hz | 0.055177 | Hz | Average shaking |
| frequency in | | | | | frequency (Hz) |
| action | | | | | |
| Amplitude | 0.061001 | (g) | 0.062601 | (g) | The farthest distance |
| | | | | | between the ACC |
| | | | | | value and the ACC |
| | | | | | average value (g) |

| | | | |
|---|---|---|---|
| Regularity | 0.88373 | 1.5668 | Standard deviation of |
| | | | each shaking period (s) |

According to the test result of this embodiment, the sensing data of the biomechanical sensing device can be used for calculation to obtain useful results for further use. The 2-norm acceleration value (ACC) is of great importance in providing information required for judgment.

Through the above and other related tests, it is known that the biomechanics assessment system of the present invention can generate a variety of sensed results in all kinds of athletic, rehabilitation, training activities, that, after suited calculation, are useful in marking informative features, classification information, or generating metadata etc., to obtain biomechanical data/information for different purposes, by using only a motion sensing elements, especially the very basic general-purpose motion sensor. The obtained biomechanical information or data can be provided with physicians, coaches and other experts for diagnosis and evaluation. The following Table II shows the correlation between the expert assessment index and the sensing data of the invented biomechanical sensing device in several body actions related to the diagnosis, treatment, and rehabilitation of Parkinson's disease.

TABLE II
Correlation between the expert assessment index and the sensing data of
the invented biomechanical sensing device in several body actions related
to the diagnosis, treatment, and rehabilitation of Parkinson's disease

| | | Position of | Expert assessment | Biomechanical sensing |
|---|---|---|---|---|
| | Item | sensing device | index | device provided data |
| 1 | Static tremor | The four | Time | |
| | | extremities | Shaking frequency | ACC toggle # |
| | | | (average) | (ACC > Threshold) |
| | | | Shaking amplitude | Max Amplitude of ACC |
| | | | (average) | |
| | | | Regularity | Toggle Interval Deviation |
| | | | (average) | |
| 2 | Kinetic tremor or | Palms of both | Time | |
| | posture tremor | hands | Shaking frequency | ACC toggle # |
| | (arms retracted) | | | (ACC > Threshold) |
| | | | Shaking amplitude | Max Amplitude of ACC |
| | | | (average) | |
| | | | Regularity | Toggle Interval Deviation |
| | | | (average) | |
| 3 | Finger tapping | Palms of both | Time/frequency | Period/Freq. |
| | for several times | hands | Amplitude | Max Amplitude of ACC |
| | (left/right) | | Regularity | Toggle Interval Deviation |
| 4 | Hand grasping | Palms of both | Time/frequency | Period/Freq |
| | several times | hands | Amplitude | Max Amplitude of ACC |
| | (left/right) | | Regularity | Toggle Interval Deviation |
| 5 | Forearm swing | palms of both | Time/frequency | Period/Freq |
| | 21 times | hands | Amplitude | Max Amplitude of Gyro |
| | (clockwise) | | Regularity | Toggle Interval Deviation |
| 6 | Foot motor ability test | Two ankles | Time/frequency | Period/Freq |
| | (Patient taps heel on | | Amplitude | Max Amplitude of ACC |
| | the ground in rapid | | Regularity | Toggle Interval Deviation |
| | succession picking up | | | |
| | entire leg) | | | |
| 7 | Raising up from | Back or palms | Complete time | Period |
| | the chair | | | |
| 8 | Posture (back | Back | Back angle | Angle |
| | angle when | | | |
| | upright) | | | |
| 9 | Gait (go/back) | Two ankles | Time/frequency | Period/Freq |
| | (left/right) | | Stride | Stride |
| | | | Cadence | Step frequency |
| | | | Height of step | Height of step |
| | | | Regularity | Deviation of Step interval |
| 10 | Posture stability | Back | Shaking frequency | GYROX toggle# |
| | (first time) | | | (GYROX > threshold) |
| | | | Amplitude | Max Amplitude of ACC |
| | | | Stabilization time | Period |
| | Posture stability | Back | Shaking frequency | GYROX toggle# |
| | (second time) | | | (GYROX > threshold) |
| | | | Amplitude | Max Amplitude of ACC |
| | | | Stabilization time | Period |

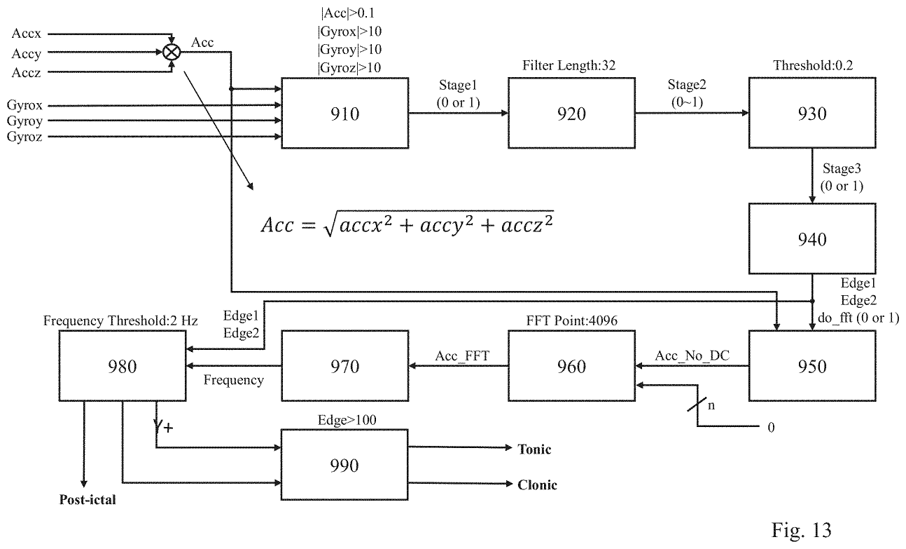

To further illustrate the possible applications of the present invention, the invented biomechanics assessment system is used to detect the epileptic seizures and history as an example. FIG. 13 is a flow chart of a method for detecting epileptic seizures and history using the biomechanics assessment system of the present invention. As shown in the figure, in step 910, the 2-norm acceleration value (ACC) of the three-axis acceleration values and the three-axis angular velocity values, both of the biomechanical sensing device are obtained. Compare the reading values with corresponding threshold values, respectively. If a value is higher than the threshold value, it is determined that the extremity where the corresponding biomechanical sensing device is worn is in tremor. In step 920, determine whether the tremor amplitude is consistent, and in step 930, determine whether the tremor amplitude exceeds a predetermined value, so as to eliminate possible false alarms. After the above steps, it can be determined that an epilepsy seizure has occurred. Next, in step 940, the clustering edge of the recording waveform is determined, and the start/transition time of the phases of the epileptic seizure is determined accordingly. In step 950, the DC component of the sensed waveform is filtered out and, in step 960, fast Fourier transform is performed on the ACC, followed by in step 970, the peak value of the frequency component is detected. In step 980, determine whether the epilepsy is in seizure. If not, it is determined as the post-seizure phase; otherwise, pulse shaping is performed in step 990, and according to the result, determine as the tonic phase or the clonic phase.

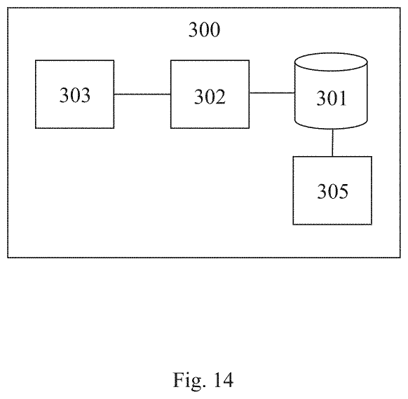

From the above tests and description, it is appreciated that the biomechanical data interpretation device 300 of the present invention serves to automatically marking features in individual sensed result files. FIG. 14 shows a block diagram of a biomechanical data interpretation device 300 applicable to the present invention. As shown in the figure, the biomechanical data interpretation device 300 of this invention is equipped with a memory device 301 for storing the biomechanical data generated by the at least one biomechanical sensing device 101-105. The biomechanical data interpretation device 300 also includes at least one functional module 302 for installing at least one biomechanical data interpretation program. Each biomechanical data interpretation program provides at least one interpretation function after operation, and each is configured to manually or automatically mark a feature to the biomechanical data in process. The marks that the biomechanical data interpretation device 300 can automatically generate include: marks representing a feature, reference information, such as type of an exercise, type of an action, sensor position, sensing time, sensing stage. The biomechanical data interpretation device 300 can also normalize the values of the sensing data. In addition, the biomechanical data interpretation device 300 may further provide an animation generating module 303, to generate animation describing the action history, based on corresponding biomechanical data, such as the multi-axial data of motion trajectory, and waveforms.

For example, after a biomechanical data interpretation program installed in the functional module 302 is executed, it can mark features on a biomechanical data file. Here, the feature may be at least one of the following features: the beginning and end of an action phase; the transition of the action phase; the beginning, the end, and the transition of an action, etc. To achieve this goal, the functional module 302 of the biomechanical data interpretation device 300 performs phase detection of the action history on a biomechanical data file in the memory device 301 or a biomechanical data file from the external, to identify a phase start address, end address, and phase transition address in the action history; all refer to the address where the signal of the start, end, and transition time is located. It then adds a mark to the address and save it back to the memory device 301.

In addition to the features related to the action history and the action phase, the biomechanical data interpretation device 300 can also perform a more detailed interpretation of the biomechanical data files. This can include determining the address/time point of the start, end, and transition of an action. In other words, for several actions that have been judged to belong to a specific action history, the type of the actions is determined. In a specific embodiment, the biomechanical data interpretation device 300 can also perform more detailed judgments, such as the time and counts of touchdowns when walking, the time and counts of take-off and landing when jumping, and so on. The related means, method and technology can be achieved by referring to the foregoing embodiments, with or without necessary modifications. After the marking is completed, the marks are recorded in the biomechanical data file and stored back to the memory device 301.

After marking the features, the biomechanical data has the information needed to provide diagnosis, treatment, rehabilitation, and training. FIGS. 3-5, FIGS. 7, 8, FIG. 9A, and FIG. 10A all show the waveforms generated by different types of biomechanical data with feature markings. The waveforms, tables or diagrams can be displayed on the display device 305 of the biomechanical data interpretation device 300, or downloaded to any computer system or the display device of the intermediate device 201-205 for users or professionals' interpretation. To this end, the biomechanical data interpretation device 300 may provide a display device 305 for retrieving one or more biomechanical data files from the memory device 301, in a format and form according to the instruction of a user.

The marking of features often involves the experience of professionals. The biomechanical data interpretation device 300 of the present invention provides a marking interface, which can be provided on the display device 305 for a professional to manually mark or manually correct marks that have been automatically made. Because most of the diagnosis, treatment, rehabilitation, and training systems will provide the function of manual marking, the relevant technical details can be omitted here.

In addition, after a biomechanical data interpretation program in the function module 302 is executed, it can automatically determine the type of exercise in any biomechanical data file. The term “types of exercise” referred one of the following athletic activities; i.e., a series of physical actions: walking, running, jumping, dancing, biking, horse riding, skiing, pulleys, and skateboarding. The judgment of the type of exercise can be achieved according to the feature judgment technology provided in the foregoing embodiments, with or without necessary modifications according to the nature of the related exercise. The commercially available exercise-related biomechanical sensing devices also provide exercise type judgment mechanisms, useful in the present invention. After the marking is made, the marks are recorded in the biomechanical data file and stored back to the memory device 301.

After a biomechanical data interpretation program in the function module 302 is executed, it can automatically determine the type of actions in any biomechanical data file. The term “types of action” referred one of the following actions: standing still, raising a hand, raising a leg, raising a palm, swinging an arm, swinging a leg, straight punch, slashing a hand, rising block, back elbow bumps, front elbow hit, side elbow hit, round kick, back kick, forward, back, turn, bend, side bend, back bend, forwards roll and backwards roll. The judgment of the action type can also be achieved according to the feature judgment technology provided in the foregoing embodiment, such as the method shown in FIG. 6, with or without necessary modifications according to the nature of the features. The commercially available exercise-related biomechanical sensing devices also provide exercise type judgment mechanisms, useful in the present invention. After the marking is made, the marks are recorded in the biomechanical data file and stored back to the memory device 301.

After a biomechanical data interpretation program in the function module 302 is executed, it can automatically mark the sensor position(s) in any biomechanical data file. The term “sensor position” refers to the position where the motion sensing element 10 is worn on the human body, and may be any of the following positions: upper left arm, upper right arm, lower left arm, lower right arm, left palm, right palm, left thigh, right thigh, left calf, right calf, left foot, right foot, head, neck, chest, back, waist and buttocks. The judgment of the wearing position can also be achieved according to the feature judgment technology provided in the foregoing embodiment, with or without necessary modifications according to the nature of the feature judgment technology. The commercially available exercise-related biomechanical sensing devices also provide exercise type judgment mechanisms, useful in the present invention. After the marking is made, the marks are recorded in the biomechanical data file and stored back to the memory device 301.

After a biomechanical data interpretation program in the function module 302 is executed, it can automatically mark the sensing time in any biomechanical data file. After a biomechanical data interpretation program in the function module 302 is executed, the sensing data of any biomechanical data file can be automatically normalized. Regarding the marking of the sensing time, it is already known to those having ordinary skills in the art. In addition, the formalization of the biomechanical data can be achieved by a skilled person, using the suited statistic theories or according to their experiences, depending on the different sensing devices, sensing objects and the combination thereof, as well as purposes of the biomechanical assessment. Detailed description thereof are thus omitted. After the time-marking or normalization is completed, the marking or normalization result is recorded in the biomechanical data file and stored back to the memory device 301.

In a preferred embodiment of the present invention, the biomechanical data interpretation device 300 is configured to recognize at least one synchronization feature in the one or more biomechanical data files, and mark a start and/or end time of display for each file, the transition frequency of the displayed content, including the data transition frequency and the frame change frequency along the time axis, according to the synchronization feature, so that the contents of a plurality of data files can be displayed simultaneously, for comparison and reference purposes. In application, the synchronization feature is preferably a time feature. According to the same or corresponding reference time, multiple sensed results obtained from different sensors or at different times and places are displayed on the same display screen in the same or different formats, making interpretation easier for professionals.

The features marking, information marking, normalization and visualization as described above can be processed without the need of a fixed processing sequence, and there are no certain steps that must be completed. There is no general rule to determine the level of detail for the phase detection of an action or an action in a action history of a biomechanical data file. Most importantly, the present invention provides a novel biomechanical assessment device and system, which can provide a variety of biomechanical assessments only by using a most basic sensing device. The invention can automatically mark features and references in the received biomechanical data, and converts the sensing data that do not have reference value into valuable information, useful for diagnosis, treatment, and rehabilitation. As a result, an exercise assessment device and system only use simple sensing devices, and do not require complex or wired sensing equipment, so they can be worn for a long time and collect biomechanical data continuously for assessment purposes. The present invention further provides a biomechanical data acquisition and processing platform, which can collect various types and a large amount of biomechanical data for long-term monitoring, training, diagnosis, and analysis.

What is claimed is:

1. A biomechanics assessment system, comprising: at least one biomechanical sensing device, at least one intermediate device and a biomechanical data interpretation device; wherein the at least one biomechanical sensing device is communicatively connected to the biomechanical data interpretation device via the at least one intermediate device;
wherein the biomechanical sensing device comprises at least one three-axis inertial sensor for sensing the movement of the biomechanical sensing device and outputting the sensing data; an interface device for receiving user input for setting at least one format for output data of the biomechanical sensing device; a wireless communication device for establishing a communication channel with the at least one intermediate device for exchange of data; and a power supply for supplying electric power to the sensor, the interface device and the wireless communication device:
wherein the intermediate device is a computer device equipped with a wireless communication function, to establish a communication channel with at least one of the plural biomechanical sensing devices for exchange of data and to establish a communication channel with the biomechanical data interpretation device to exchange data;
wherein the biomechanical data interpretation device is provided with a memory device for storing the biomechanical data generated by the at least one biomechanical sensing device; and
wherein the biomechanical data interpretation device is provided with at least one biomechanical data interpretation program, each being configured to perform at least one of the processing on the biomechanical data: marking a feature, marking reference information, including one of physical activity, type of action, sensor position, sensing time and stage of sensing/action; and to perform normalization on the biomechanical data.

2. The biomechanics assessment system according to claim 1, wherein the biomechanical data interpretation device is embedded in the intermediate device, in form of an application software.

3. The biomechanics assessment system according to claim 1, wherein the biomechanical data interpretation device is embedded in a server computer connected to the Internet in form of an application software

4. The biomechanics assessment system according to claim 1, wherein the biomechanical assessment system comprises a plurality of biomechanical sensing devices, a plurality of intermediate devices, and at least one biomechanical data interpretation device, wherein at least one of the plural biomechanical sensing devices is communicatively connected to the biomechanical data interpretation device via at least one of the plural intermediate devices, and wherein the biomechanical data interpretation device is installed in one server computer and is connected to the plurality of intermediate device via the Internet.

5. The biomechanics assessment system according to claim 1, wherein the intermediate device provides a setting interface, preferably a graphical setting interface, for the user to input setting parameters, and to send them to the biomechanical sensing device to change a parameter of the biomechanical data sensing device, including at least one format of output sensing data.

6. The biomechanics assessment system according to claim 5, wherein the biomechanical sensing device is configured to continuously output the sensing data via the wireless communication device in the at least one format for a predetermined time.

7. The biomechanics assessment system according to claim 5, wherein the biomechanical sensing device further comprises a memory device for storage of the sensing data of the inertial sensor, wherein the biomechanical sensing device is configured to continuously store the sensing data in the memory device in the at least one format for a predetermined time.

8. The biomechanics assessment system according to claim 7, further comprising a display device for retrieving one or more biomechanics data file from the memory device of the biomechanical data interpretation device according to a user's instruction, and displaying the requested information in the at least one format selected by the user.

9. The biomechanics assessment system according to claim 1, wherein the biomechanical sensing device further comprises a gyroscope and/or a three-axis magnetometer.

10. The biomechanics assessment system according to claim 1, wherein the intermediate device is configured to supply or transmit the sensing data sent by the at least one biomechanical sensing device to the biomechanical data interpretation device.

11. The biomechanics assessment system according to claim 1, wherein the interface device of the biomechanical sensing device is built in the intermediate device

12. The biomechanics assessment system according to claim 2, wherein the biomechanical data interpretation program is executed on the server computer, to mark a feature on a biomechanical data file, wherein the feature to be marked comprises be at least one of the group consisted of the following features: a beginning and an end of a physical activity; a transition of a stage of the physical activity; a beginning, an end, and a transition of a type of a physical action; and generation of a movement trajectory.

13. The biomechanics assessment system according to claim 4, wherein the biomechanical data interpretation program is executed on the server computer, to mark a feature on a biomechanical data file, wherein the feature to be marked comprises be at least one of the group consisted of the following features: a beginning and an end of a physical activity; a transition of a stage of the physical activity; a beginning, an end, and a transition of a type of a physical action; and generation of a movement trajectory.

14. The biomechanics assessment system according to claim 2, wherein the biomechanical data interpretation program is executed on the server computer, to identify a type of physical activity for a biomechanical data file, wherein the type to be identified comprises be at least three of the group consisted of the following types: walking, running, jumping, dancing, biking, horse riding, skiing, skating, and skateboarding.

15. The biomechanics assessment system according to claim 4, wherein the biomechanical data interpretation program is executed on the server computer, to identify a type of physical activity for a biomechanical data file, wherein the type to be identified comprises be at least three of the group consisted of the following types: walking, running, jumping, dancing, biking, horse riding, skiing, skating, and skateboarding.

16. The biomechanics assessment system according to claim 2, wherein the biomechanical data interpretation program is executed on the server computer, to identify a type of action for a biomechanical data file, wherein the type to be identified comprises be at least three of the group consisted of the following types: standing still, raising a hand, raising a leg, raising a palm, swinging an arm, swinging a leg, straight punch, slashing a hand, rising block, back elbow bumps, front elbow hit, side elbow hit, round kick, back kick, forward, back, turn, bend, side bend, back bend, forwards roll and backwards roll.

17. The biomechanics assessment system according to claim 4, wherein the biomechanical data interpretation program is executed on the server computer, to identify a type of action for a biomechanical data file, wherein the type to be identified comprises be at least three of the group consisted of the following types: standing still, raising a hand, raising a leg, raising a palm, swinging an arm, swinging a leg, straight punch, slashing a hand, rising block, back elbow bumps, front elbow hit, side elbow hit, round kick, back kick, forward, back, turn, bend, side bend, back bend, forwards roll and backwards roll.

18. The biomechanics assessment system according to claim 2, wherein the biomechanical data interpretation program is executed on the server computer, to mark a sensor position for a biomechanical data file, wherein the position to be marked comprises be at least four of the group consisted of the following positions: upper left arm, upper right arm, lower left arm, lower right arm, left palm, right palm, left thigh, right thigh, left calf, right calf, left foot, right foot, head, neck, chest, back, waist and buttocks.

19. The biomechanics assessment system according to claim 4, wherein the biomechanical data interpretation program is executed on the server computer, to mark a sensor position for a biomechanical data file, wherein the position to be marked comprises be at least four of the group consisted of the following positions: upper left arm, upper right arm, lower left arm, lower right arm, left palm, right palm, left thigh, right thigh, left calf, right calf, left foot, right foot, head, neck, chest, back, waist and buttocks.

20. The biomechanics assessment system according to claim 2, wherein the biomechanical data interpretation program is executed on the server computer, to sensing time for a biomechanical data file and/or to normalize the sensing data of a biomechanical data file.

21. The biomechanics assessment system according to claim 4, wherein the biomechanical data interpretation program is executed on the server computer, to sensing time for a biomechanical data file and/or to normalize the sensing data of a biomechanical data file.

22. The biomechanics assessment system according to claim 4, wherein the biomechanical data interpretation device is configured to mark at least one synchronization feature in one or more biomechanical data files, and mark a start and/or end time of display for each file, a transition frequency of displayed content, including a data transition frequency and a frame change frequency along the time axis, according to the synchronization feature.
